(19) United States
(12) Patent Application Publication
(10) Pub. No.: US 2015/0150817 A1
(43) Pub. Date: Jun. 4, 2015

(54) FILM COMPOSITION FOR HARD CAPSULE SHELLS

(71) Applicant: Dow Global Technologies LLC, Midland, MI (US)

(72) Inventor: Bettina Hoelzer, Walsrode (DE)

(73) Assignee: DOW GLOBAL TECHNOLOGIES LLC, Midland, MI (US)

(21) Appl. No.: 14/415,614

(22) PCT Filed: Jul. 12, 2013
(86) PCT No.: PCT/US13/50217

§ 371 Date: Jan. 19, 2015

Related U.S. Application Data
(60) Provisional application No. 61/674,628, filed on Jul. 23, 2012.

(51) Int. Cl. A61K 9/48; A61K 47/38; A61K 47/18; C08J 5/18

(52) U.S. Cl. CPC A61K 9/4816; C08J 5/18; A61K 47/38; A61K 47/18; C08J 2301/28

(57) ABSTRACT

A film-forming composition comprising (a) a cellulose derivative such as HPMC and (b) a urea additive selected from urea, urea derivatives and mixtures thereof; the use of the film-forming composition as a shell material for hard capsules; and films and hard capsules prepared from the film-forming composition.

The present invention relates to a film-forming composition comprising a cellulose derivative as a base material and to films and hard capsules which are prepared from the composition and show improved disintegration behavior.

Hard capsules are a popular dosage form for oral administration of drugs and —due to ease of swallowing—also for nutritional and food supplements. Gelatin is the traditionally used shell material for hard capsules. However, as it is animal-derived it is associated with bovine spongiform encephalopathy (BSE) and transmissible spongiform encephalopathy (TSE). The use of cellulose ethers, especially hydroxypropyl methylcellulose (HPMC), for hard capsules is an attractive alternative to gelatin capsules because of its vegetable source. A major impediment for broad use of HPMC capsules for pharmaceutical applications is the slower disintegration of HPMC capsules versus gelatin capsules (M. M. A. Tabakha, J. Pharm Parmaceut Sci, 2010, 13, 428-442; M. S. Ku, W. Li, W. Dulin, F. Donahue, D. Cade, H. Benameur, K. Hutchison, Int. Journal of Pharmaceutics, 2010, 386, 30-41 and “Mechanical, Permeation, and Disintegration Behavior of Films Based on Hypromellose and its Blends”, Jin Zhao et al, Poster presented at the 2009 Annual Meeting and Exposition of the American Association of Pharmaceutical Scientists, Los Angeles, Calif., Nov. 8-12, 2009). Several additives to enhance the disintegration of HPMC capsules have been reported in the prior art.

W 00/69418 describes enhanced dissolution rates of cellulose ether capsules by addition of 0.1-15 weight percent of an organic acid, preferably citric acid. The resulting cellulose ether composition had a pH value of 6 or less. The low pH value will shorten shelf-life of capsules filled with acid sensitive prodrugs which forms the actual bioavailable drug in the stomach at low pH.

WO 2006/082842 relates to a hard capsule produced by adding a sugar alcohol to the cellulose ether to improve capsule solubility and molding processability. JP 2010-270039 concerns a hard capsule containing HPMC and monosaccharide, disaccharide and/or starch for improved water solubility or hardness. However, these sugar additives enable rapid microbiological growth.

EP 1 757 310 A1 is directed to a solubility-improved hard capsule containing a water-soluble cellulose derivative as a base material and one or two compound(s) selected from polyvinylpyrrolidones, copolymers of vinylpyrrolidone with vinyl acetate, and polyethylene glycols. The use of synthetic copolymer additives such as polyvinylpyrrolidones in edible products is not preferred due to the artificial source.

It is thus the object of the present invention to provide a vegetable-derived film-forming composition suitable as shell material for hard capsules which exhibits improved disintegration behavior vis-à-vis HPMC hard capsules but avoids the drawbacks of the prior art.

The object is met by a film-forming composition comprising (a) a cellulose derivative and (b) a urea additive selected from urea, urea derivatives and mixtures thereof.

The present invention is also directed to the use of the film-forming composition as a shell material for hard capsules, a tablet coating or an excipient for pharmaceutical agents and medicaments and to a hard capsule comprising the film-forming composition.

The cellulose derivatives (a) for use in the present invention are preferably water-soluble, i.e. they have a solubility in water of at least 1 gram, more preferably at least 2 grams, most preferably at least 5 grams in 100 grams of distilled water at 25° C. and 1013 hPa. Typically, the cellulose derivative (a) is a cellulose ether, preferably an alkyl cellulose, hydroxyalkyl cellulose or hydroxyalkyl alkylcellulose. This means that in the cellulose ether at least a part of the hydroxyl groups of the cellulose backbone at the 2-, 3- and 6-positions of the anhydroglucose units are substituted by alkoxy groups, preferably C₁ to C₄ alkoxy groups, or hydroxyalkoxy groups, preferably C₁ to C₄ hydroxyalkoxy groups, or a combination of alkoxy and hydroxyalkoxy groups. The hydroxyalkoxy groups are typically hydroxymethoxy, hydroxyethoxy and/or hydroxypropoxy groups. Hydroxyethoxy and/or hydroxypropoxy groups are preferred. Typically one or two kinds of hydroxyalkoxy groups are present in the cellulose ether. Preferably a single kind of hydroxyalkoxy group, more preferably hydroxypropoxy, is present. The alkoxy groups are typically methoxy, ethoxy and/or propoxy groups. Methoxy groups are preferred.

Examples of cellulose ethers for use in the present invention are alkylcelluloses, such as methylcellulose (MC); hydroxyalkylcelluloses, such as hydroxyethylcellulose, hydroxypropylcellulose, and hydroxybutylcellulose; and hydroxyalkyl alkylcelluloses, such as hydroxyethyl methylcellulose (HEMC), hydroxymethyl ethylcellulose, ethyl hydroxyethylcellulose (EHEC), hydroxypropyl methylcellulose (HPMC), hydroxypropyl ethylcellulose, hydroxybutyl methylcellulose (HBMC), and hydroxybutyl ethylcellulose; and those having two or more hydroxyalkyl groups, such as hydroxyethylhydroxypropyl methylcellulose. More preferably, the cellulose ether is selected from hydroxypropyl methylcellulose, hydroxyethyl methylcellulose, methylcellulose and mixtures thereof. Most preferably, the cellulose ether is hydroxypropyl methylcellulose.

The term “cellulose derivative (a)” as used herein also implies mixtures of different types of cellulose derivatives including mixtures of the exemplary and preferred cellulose ethers mentioned before.

For hydroxyalkyl celluloses or hydroxyalkyl alkylcelluloses the degree of the substitution of hydroxyl groups at the 2-, 3- and 6-positions of the anhydroglucose units by hydroxyalkoxy groups is expressed by the molar substitution of hydroxyalkoxy groups (MS). The MS is the average number of moles of hydroxyalkoxy groups per anhydroglucose unit in the cellulose ether. It is to be understood that during the hydroxyalkylation reaction the hydroxyl group of a hydroxyalkoxy group bound to the cellulose backbone can be further etherified by an alkylation agent, e.g. a methylation agent, and/or a hydroxyalkylation agent. Multiple subsequent hydroxyalkylation etherification reactions with respect to the same carbon atom position of an anhydroglucose unit yields a side chain, wherein multiple hydroxyalkoxy groups are covalently bound to each other by ether bonds, each side chain as a whole forming a hydroxyalkoxy substituent to the cellulose backbone. The term “hydroxyalkoxy groups” thus has to be interpreted in the context of the MS as referring to the hydroxyalkoxy groups as the constituting units of hydroxyalkoxy substituents, which either comprise a single hydroxyalkoxy group or a side chain as outlined above, wherein two or more hydroxyalkoxy units are covalently bound to each other by ether bonding. Within this definition it is not important whether the terminal hydroxyl group of a hydroxyalkoxy substituent is further alkylated, e.g. methylated, or not; both alkylated and non-alkylated hydroxyalkoxy substituents are included for the determination of MS. The cellulose ethers for use in the present invention preferably have an MS from 0.05 to 1.00, preferably from 0.07 to 0.80, more preferably from 0.08 to 0.70, most preferably from 0.09 to 0.60, and particularly from 0.10 to 0.50.

For alkyl celluloses and hydroxyalkyl alkylcelluloses the average number of hydroxyl groups substituted by alkoxy groups, such as methoxy groups, per anhydroglucose unit, is designated as the degree of substitution of alkoxy groups (DS). In the above-given definition of DS, the term “hydroxyl groups substituted by alkoxy groups” is to be construed within the present invention to include not only alkylated hydroxyl groups directly bound to the carbon atoms of the cellulose backbone, but also alkylated hydroxyl groups of hydroxyalkoxy substituents bound to the cellulose backbone. The cellulose ethers for use in the present invention preferably have a DS from 1.1 to 2.5, more preferably from 1.2 to 2.3, most preferably from 1.3 to 2.2 and particularly from 1.4 to 2.1.

The degree of substitution of alkoxy groups and the molar substitution of hydroxyalkoxy groups can be determined by Zeisel cleavage of the cellulose ether with hydrogen iodide and subsequent quantitative gas chromatographic analysis (G. Bartelmus and R. Ketterer, Z. Anal. Chem., 286 (1977) 161-190).

The viscosities of 2% by weight aqueous solutions of cellulose ethers for use in the present invention preferably range from 2.4 to 200 mPa·s, more preferably from 2.5 to 100 mPa·s, even more preferably from 2.7 to 50 mPa·s, most preferably from 2.8 to 30 mPa·s, and in some embodiments from 3 to 15 mPa·s, determined in a 2% by weight solution in water at 20° C. The 2% weight solution viscosities referred to within the present application are all determined at 20° C. according to ASTM D2363-79 (Reapproved 2006) with an Ubbelohde tube viscometer. The solution viscosity reflects the molecular weight of the cellulose derivative.

In preferred embodiments the present film-forming composition comprises HPMC having an average degree of substitution DS_{methyl} of from 1.1 to 2.5, more preferably of from 1.2 to 2.3, even more preferably from 1.3. to 2.2, still more preferably from 1.4 to 2.1, and most preferably from 1.5 to 2.0 and a molar degree of substitution MS_{hydroxypropyl} of from 0.05 to 1.00, more preferably of from 0.07 to 0.80, even more preferably from 0.08 to 0.70, still more preferably of from 0.09 to 0.60, most preferably from 0.10 to 0.50, and particularly from 0.10 to 0.40. Typically, viscosities of 2% by weight aqueous HPMC solutions at 20° C. range from 2.4 to 200 mPa·s, preferably from 2.5 to 100 mPa·s, more preferably from 2.7 to 50 mPa·s, even more preferably from 2.8 to 30 mPa·s, most preferably from 3 to 15 mPa·s, and in certain embodiments the 2% by weight viscosity is about 5 mPa·s. Examples of commercially available HPMCs that are useful in the present invention include Methocel F4 and F5 (27.0-30.0% methoxyl, 4.0-7.5% hydroxypropoxyl, respectively: DS_{methyl}=1.6-1.9; MS_{hydroxypropyl}=0.1-0.2; 2% by weight viscosity=3-6 mPas) and Methocel E5 and E6 (28.0-30.0% methoxyl, 7.0-12.0% hydroxypropoxyl, respectively: DS_{methyl}=1.1-2.1; MS_{hydroxypropyl}=0.18-0.34; 2% by weight viscosity=4-7 mPas), each available from The Dow Chemical Company, Midland, U.S.A.

Component (b) of the film-forming composition of the present invention, the urea additive, is selected from urea, a urea derivative, a mixture of urea and at least one urea derivative, or a mixture of different urea derivatives. “Urea derivative” within the meaning of the present invention designates any derivative of urea formed by the replacement of 1 to 4 hydrogen atoms of the NH₂ groups. It specifically includes 1- (or N—), 1,1- (or N,N—), 1,1,3- (or N,N,N′—), or 1,1,3,3- (or N,N,N′,N′—) substituted urea. Exemplary substituents include alkyl groups such as C₁ to C₄ alkyl groups, e.g. methyl, ethyl, propyl, butyl; aryl groups such as C₅ to C₁₀ aryl groups, e.g. phenyl, and cycloalkyl groups such as C₃ to C₆ cycloalkyl groups, e.g. cyclopentyl, cyclohexyl. The most preferred urea additive for use in the present invention is urea. Urea is especially useful in film-forming composition for use as a shell material for hard capsules, a tablet coating or an excipient material because urea is approved for food and pharmaceutics.

Typically, the cellulose derivative (a) and the urea additive (b) are present in the inventive film-forming composition in a weight ratio (a):(b) of from 99:1 to 80:20, preferably of from 95:5 to 90:10. The amount of cellulose derivative (a) means the total amount of all cellulose derivatives present in the film-forming composition. The amount of urea additive (b) means the total amount of urea and all urea derivatives present in the film-forming composition.

It is surprising that a film-forming composition wherein a urea additive (b) has been added to a cellulose derivative (a) results in a film exhibiting improved disintegration rates under stomach conditions as compared to a comparable film prepared in absence of the urea additive (b). Since the urea additive (b) has basic character it does not shorten shelf-life of capsules filled with acid sensitive prodrugs.

In the present invention, additives other than the urea additive (b) may be optionally formulated to the cellulose derivative (a). Examples of the additives include gelling agents, plasticizers, gelling auxiliary agents, colorants, pigments, sugars, sweeteners, and flavoring agents.

The film-forming composition of the present invention may optionally comprise an additional gelling agent other than the cellulose derivative (a). Typical gelling agents are polysaccharide hydrocolloids such as natural gums including vegetable gums and gums derived from algae and bacteria. Representative hydrocolloids are disclosed in WO 98/27151. Suitable examples include carrageenan, pectin, curdlan, agar, gellan gum, tamarind seed polysaccharide, alginates, guar gum, locust bean gum, tara gum, gum arabic, ghatti gum, arabian (araban), xanthan, starch, galactomannan, funoran, pullulan, and dextran. The gelling agent may be added in an amount of preferably up to 3.0% by weight (0 to 3% by weight), based on the weight of the solids of the film-forming composition. If used, the amount of the gelling agent is typically 0.1 to 3.0% by weight, preferably 0.25 to 2.5% by weight, and more preferably 0.5 to 2.0% by weight, based on the weight of the solids of the film-forming composition. However, in preferred embodiments the present film-forming composition does not contain a further gelling agent in addition to the cellulose derivative (a).

Examples of the optional plasticizers for use in the present film-forming composition include triethyl citrate, triacetin, and Poylsorbat 80 (Tween® 80), glycerol, sorbitol, glycol, polyethylene glycol, dioctyl-sodium sulfosuccinate, 1,2-propylenglycol, and mono-, di- or triacetates of glycerols. However, in preferred embodiments the present film-forming composition does not contain a plasticizer.

As optional gelling auxiliary agents, there may be mentioned, for example, potassium chloride, ammonium chloride, ammonium acetate, and calcium chloride. However, in preferred embodiments the present film-forming composition does not contain a gelling auxiliary agent.

Typically, the total amount of cellulose derivative (a) and the urea additive (b) makes up 40 to 100% by weight of the solids of the film-forming composition, preferably 60 to 100% by weight, more preferably 80 to 100% by weight, even more preferably 90 to 100% by weight, and most preferably 94 to 100% by weight.

The film-forming composition according to the present invention can be processed to films, e.g. cast into films or formed to shells by dip coating. Accordingly, the present invention is also directed to a film prepared from the film-forming composition.

If the film-forming composition is to be processed to a film it typically comprises water and more preferably the film-forming composition is in the form of an aqueous solution. In these cases the film-forming composition preferably comprises 60 to 95% by weight, more preferably 65 to 90% by weight, even more preferably 70 to 88% by weight, still more preferably 75 to 85% by weight, and most preferably 77 to 83% by weight of water (each based on the total weight of the film-forming composition), the remainder being solids. In some embodiments the aqueous film-forming composition has a pH value of greater than 7, preferably equal to or greater than 7.2, more preferably equal to or greater than 7.3, and most preferably equal to or greater than 7.4.

The dried film prepared from the film-forming composition according to the present invention has a considerably reduced water content. In these cases the film-forming composition is free of water or has a maximum water content of typically no more than 10% by weight, preferably no more than 8% by weight, and most preferably no more than 6% by weight, the remainder being solids. In other words a dried film prepared from the film-forming composition typically comprises 0 to 10% by weight, preferably 0 to 8% by weight, and more preferably 0 to 6% by weight, such as 3 to 6% by weight, of water (each based on the total weight of the film), the remainder being solids.

In some embodiments the film-forming composition of the present invention may comprise a water/alcohol mixture instead of water. In these cases a part of the water, such as present in the preferred amounts mentioned above, is replaced by an alcohol, for example methanol, ethanol, propanol, or mixtures thereof.

The present film-forming composition may be used as a shell material for hard capsules, a tablet coating or an excipient for pharmaceutical agents and medicaments in capsules and tablets.

The present invention also concerns a hard capsule comprising a shell comprising the film-forming composition, i.e. a shell prepared from the present film-forming composition. Typically, the hard capsule comprises a drug or a nutritional and food supplement surrounded by the shell material made of the inventive film-forming composition. The hard capsules are preferably two-piece hard capsules. Those are typically manufactured by dipping hot metal pins or bars in a cold, aqueous coating solution of the film-forming composition. The solution thermally gels on the pins and water evaporates during a drying step to form thin film layers of dried cellulose ether around the hot pins. The thin films take the form of caps and bodies, which are then removed from the pins. Caps are mated with bodies to form capsules. Analogous processes exist wherein cold pins are dipped in a hot, aqueous solution of the film-forming composition. Both processes are within the scope of the present invention. Processes for making capsules are also disclosed in U.S. Pat. Nos. 3,617,588; 4,001,211; 4,917,885; and 5,756,036.

Some embodiments of the invention will now be described in detail in the following examples wherein all parts and percentages are by weight unless otherwise specified.

### EXAMPLES

Film Preparation

METHOCEL™ F5 Premium LV Hydroxypropyl Methylcellulose (27.0-30.0% methoxyl, 4.0-7.5% hydroxypropoxyl, respectively: DS_{methyl}=1.6-1.9; MS_{hydroxypropyl}=0.1-0.2; 2% by weight viscosity=about 5 mPa·s) was blended with different amounts of urea (see Table). 20% by weight solutions in water were prepared using the reference material (HPMC only) and the HPMC blends according to the following procedure. The HPMC or HPMC blends were quickly dispersed into an 800-mL beaker equipped with a three-blade shaft containing boiling deionized water (>90° C.). The rotation rate during the addition of powder was set to maintain a vortex that continuously drew the powder into the water (>400 rpm). After powder addition, the solutions were covered and allowed to stir (400 rpm) for an additional 3 hours at room temperature. Each solution was then stored in a refrigerator for 6 hours and further in a vacuum oven (at about 0.05 MPa) at room temperature for 1 hour to eliminate air bubbles. Films (about 15 cm×8 cm) were cast on glass plates at room temperature by hand using a casting bar (1000 μm wet film thickness, from LAU GmbH, 58675 Hemer, Germany). The films were allowed to air dry in a constant temperature room (22° C. and 50% relative humidity) for two days, removed, and annealed for an additional day before any film properties were measured. The films prepared from these solutions had film thicknesses which were very similar (133-136 μm).

Film Disintegration

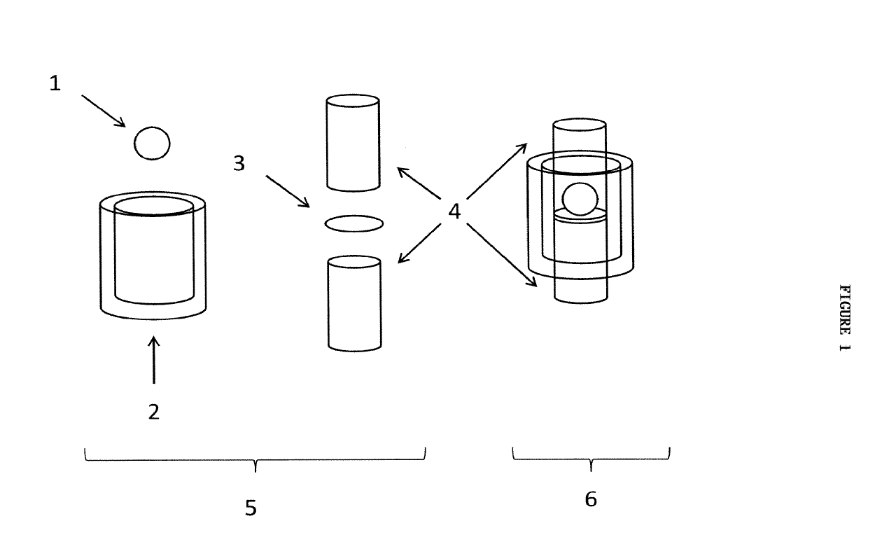

Film disintegration was tested using a ball sample holder in conjunction with the disintegration tester ZT72 from ERWEKA GmbH, 63150 Heusenstamm, Germany. The instrument is equipped with a rigid basket-rack assembly supporting six cyclindrical glass tubes 77.5 mm long, 21.5 mm in internal diameter as described in the European Pharmacopoeia (Seventh edition, 2011, Disintegration of Tablets and Capsules). The assembly was suspended in KCl/HCl buffer (pH 1.2) The film was subjected to a controlled stress via a steel ball to simulate stomach conditions. FIG. 1 shows a schematic of the sample holder. In each cyclindrical glass tube of the disintegration tester one sample holder with a film sample was placed and fixed. After the assembly was suspended in the buffer the time was detected when the steel ball fell down. The disintegration times shown in the Table were the averages of 10 measurements.

Description of FIG. 1:
1: Stainless steel ball (weight 1.04 g)
2: Outside sleeve
3: Film sample
4: Film sample holder
5: Before assemble
6: After assemble

TABLE
Disintegration times in KCl/HCl buffer medium
(pH = 1.2) at 37° C.

| | Weight Ratio | pH of Casting | Disintegration |
|---|---|---|---|
| Example No. | HPMC/urea | Solution | Time/s |
| Reference Example | only HPMC | 7.0 | 181 |
| Example 1 | 95:5 | 7.4 | 156 |
| Example 2 | 90:10 | 7.7 | 132 |

It is evident from the results above that films prepared from the inventive film-forming composition show improved disintegration under simulated stomach conditions.

1. A film-forming composition comprising (a) a cellulose derivative selected from hydroxypropyl methylcellulose, methylcellulose, hydroxyethyl methylcellulose, and mixtures thereof and (b) urea, wherein the total amount of the cellulose derivative (a) and the urea (b) constitutes 90 to 100% by weight of the solids of the film-forming composition.

2. The film-forming composition of claim 1 comprising the cellulose derivative (a) and the urea (b) in a weight ratio (a):(b) of from 99:1 to 80:20.

3. The film-forming composition of claim 1, wherein the total amount of the cellulose derivative (a) and the urea (b) constitutes 94 to 100% by weight of the solids of the film-forming composition.

4. The film-forming composition of claim 1, wherein the cellulose derivative is hydroxypropyl methylcellulose.

5. The film-forming composition of claim 1 further comprising 0 to 10% by weight of water, based on the total weight of the film-forming composition.

6. The film-forming composition of claim 1 further comprising 60 to 95% by weight of water, based on the total weight of the film-forming composition.

7. (canceled)

8. A film prepared from the film-forming composition according to claim 1.

9. A hard capsule comprising a shell prepared from a film-forming composition comprising (a) a cellulose derivative and (b) urea.

10. The hard capsule of claim 9 wherein the film-forming composition comprises the cellulose derivative (a) and the urea (b) in a weight ratio (a):(b) of from 99:1 to 80:20.

11. The hard capsule of claim 9, wherein the total amount of the cellulose derivative (a) and the urea (b) constitutes 40 to 100% by weight of the solids of the film-forming composition.

12. The hard capsule of claim 9, wherein the cellulose derivative (a) is a cellulose ether.

13. The hard capsule of claim 12, wherein the cellulose ether is selected from hydroxypropyl methylcellulose, methylcellulose, hydroxyethyl methylcellulose, and mixtures thereof.

14. The film-forming composition of claim 13, wherein the cellulose ether is hydroxypropyl methylcellulose.

15. The hard capsule of claim 9 further comprising a drug or a nutritional and food supplement.

16. The film-forming composition of claim 1 comprising the cellulose derivative (a) and the urea (b) in a weight ratio (a):(b) of from 95:5 to 90:10, the cellulose derivative being hydroxypropyl methylcellulose.

17. The hard capsule of claim 10 wherein the total amount of the cellulose derivative (a) and the urea (b) constitutes 40 to 100% by weight of the solids of the film-forming composition.

18. The hard capsule of claim 17 wherein the cellulose derivative (a) is a cellulose ether.

19. The hard capsule of claim 18 wherein the cellulose ether is selected from hydroxypropyl methylcellulose, methylcellulose, hydroxyethyl methylcellulose, and mixtures thereof.
